(19) United States
(12) Patent Application Publication
(10) Pub. No.: US 2011/0160302 A1
(43) Pub. Date: Jun. 30, 2011

(54) DEVICE AND METHODS FOR TREATMENT AND PREVENTION OF TENDON INJURIES

(76) Inventor: Andrew Cabot, Boston, MA (US)

(21) Appl. No.: 12/452,195

(22) PCT Filed: Jun. 16, 2008
(86) PCT No.: PCT/US2008/007558

§ 371 Date: Mar. 18, 2011

Related U.S. Application Data
(60) Provisional application No. 60/936,113, filed on Jun. 18, 2007.

(51) Int. Cl. A61K 31/21; A61K 31/045; A61P 43/00; A61F 7/00

(52) U.S. Cl. 514/509; 514/729; 607/108

(57) ABSTRACT

The present invention relates to treatment and prevention devices and regimens for tendon injury and damage. The invention further provides compositions comprising therapeutic agents and the use of such compositions in methods of treating disorders, injuries and conditions beneficially treated by these agents, particularly those relating to the management of tendon damage.

### CROSS-REFERENCE TO RELATED APPLICATIONS

This application claims benefit of U.S. application Ser. No. 60/936,113, filed Jun. 18, 2007. The contents of which are incorporated by reference in their entirety.

### TECHNICAL FIELD OF THE INVENTION

The present invention relates to treatment and prevention devices and regimens for tendon injury and damage. The invention further provides compositions comprising therapeutic agents and the use of such compositions in methods of treating disorders, injuries and conditions beneficially treated by these agents, particularly those relating to the management of tendon damage.

### BACKGROUND OF THE INVENTION

The increasing aging of the “baby boomer” generation, increasing numbers are now reaching their 40s, 50s and 60s. On the one hand, there has been, particularly in the United States, increasing incidence of obesity, thus resulting in increased strain on the body and body parts due to increased weight load bearing. On the other hand, concurrent with this, an increasing number of these adults continue to actively participate in physical activities (e.g., sports, recreation, outdoor activities) throughout life compared to generations before. There is also an increase in children and young adults participating both at an earlier age, and at a substantially more involved level in sports and other physical activity. With this significant increase in activity, it is not surprising that the rate of injury related to such activity is also on the increase. In particular, damage (both traumatic injury as well as general wear and tear) to tendons in the body (e.g., Achilles, tendons associated with rotator cuff, bicep tendon, tennis/golfer's elbow, patella tendon, hamstring, groin, tricep, ITB, hand/wrist tendons, DeQuervain's Tenosynovitis) is increasing, particularly relating to athletic activity. Typical therapeutic regimens for such tendon injury include, for example, physical therapy, anti-inflammatories, steroids, acupuncture, deep tissue/sports massage, chiropractic, cortisone injection, immobilization, rest/cessation, of activity, alternative therapies (e.g., herbal, reichi, shiatsu, myofascial release, ART, energy healing, Feldenkrias, reflexology and the like). However, such existing therapies suffer from limitations, including for example, effectiveness (short term, long term), compliance, ease of administration, high cost, length of time for treatment or rest, adverse side effects from medicines, side effects of rest (e.g., atrophy, tissue degeneration, etc.). As such, novel approaches and new treatment or prevention regimens are desired.

### SUMMARY OF THE INVENTION

The present invention provides devices, compositions, and methods of treating and preventing disorders, injuries or symptoms thereof by administration of the devices and compositions thereof The invention also relates to methods of protecting body parts from physical insult and injury comprising use of the devices/compositions of the invention as well.

### DETAILED DESCRIPTION OF THE INVENTION

In one aspect, the invention provides a device that comprises a substrate and a therapeutic agent. The device is useful for application to a body part in order to provide healing function, increased comfort or pain relief, or injury protection (e.g., protecting from injury or re-injury) to the body part. In particular, the device is useful in methods of treating an injured or damaged body part, in methods of preventing injury or deterioration of the body part, or in methods of providing desired function of the body part such that superior performance of the body part is achieved, or potential injury (or re-injury) or damage to such body part is minimized. In aspects, the device and methods of using the device provide increased mobility and movement of the subject, increased comfort in undertaking physical activity (particularly at the injured or strained body part), or provides the ability for the subject to perform and participate in activity for increased durations of time compared to without the device or application thereof. In other aspects, the device or application thereof to a subject reduces the risk of re-injury of the body part, particularly during participation in physical activity. In one aspect the subject tendon is healthy or not injured. In another aspect, the subject tendon is or was injured.

In one aspect, the invention relates to a substrate comprising an agent that induces blood flow, circulation, elasticity, flexibility, or other normal function to the body part (or decreased loss or reduction of any respective function). Such body part can be in an injured or other state of disrepair, or can be in a normal state but subject to minimal (e.g., unnoticed wear and tear to the subject) insult that can over time lead to more extensive injury. The substrate can be applied to the body part that is already damaged or subject to potential injury. In such respect, the device or method thereof can contribute to healing of the injured or stressed body part.

The terms “ameliorate” and “treat” are used interchangeably and both mean decrease, suppress, attenuate, diminish, arrest, or stabilize the development or progression of an injury, damage or disorder (e.g., disease or disorders, or symptom thereof, such as those delineated herein).

The term “athletic activity” means any physical activity or physical exertion of a body part (i.e. a tendon) performed in participation in work, sport or recreation.

The term “tendon injury” meansany injury relating to an overloading of a tendon or muscle tendon complex causing inflammation, tearing, scarring, or prompting other degereative changes to the tendon, including but not limited to, tendonitis, tenosynovitis, tendinopathy, tendinosis, strain/tear or rupture.

By “disease” or “disorder” is meant any condition or disorder that damages or interferes with the normal function of a cell, tissue, or organ; including those delineated herein.

In this disclosure, “comprises,” “comprising,” “containing” and “having” and the like can have the meaning ascribed to them in U.S. Patent law and can mean “includes,” “including,” and the like; “consisting essentially of”or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

In general, an “effective amount” of a biologically and/or pharmacologically and/or therapeutically active agent is an amount sufficient to achieve a desired biological and/or pharmacological and/or therapeutic effect when delivered to a cell or organism (e.g., subject body part) according to a selected administration form, route, and/or schedule. As will be appreciated by those of ordinary skill in this art, the absolute amount of a particular agent that is effective may vary depending on such factors as the desired biological/functional/therapeutic endpoint, the agent to be delivered, the target body part, etc. Those of ordinary skill in the art will further understand that an “effective amount” may be administered in a single dose, or may be achieved by administration of multiple doses. For example, an effective amount of a therapeutic agent may be an amount sufficient to achieve one or more of the following: (i) inhibit body part damage; (ii) reduce the severity of or prevent one or more symptoms or signs of body part damage; (iii) significantly reduce the risk of recurrence of a body part injury; (iv) significantly reduce the risk of a further body part injury; (v) induce repair or recovery of body part function due to body part damage, etc.

The term “microtrauma””, as used herein, refers to a small lesion(s), and with respect to tendoin injury, refers to the overloading of the musculotendinous unit causing microstrains/tears of the tissue. Microtrauma induces inflammation, which results in degeneration of the tendon.

The term “repetitive stress injury”, as used herein, refers to a cumulative effect of microtrauma and is marked by the process in which repeated application of high intensity loading force to the muscle and tendon complex leads to one or more of inflammation, tearing or degeneration.

As used herein, agent or entity is “isolated” if it is separated from at least some materials or components with which it is associated in nature or when initially generated. In general, such separation involves activity of the hand of man. In aspects, the chemical compounds delineated herein are isolated.

The term “mobility” refers to the ease of moving from one point to another. In regard to a tendon, “mobility” is in reference to movement of the tendon in the relevant body part for that physical activity for which the tendon/ body part are associated with.

A “subject”, as used herein, is an individual to whom an agent is to be delivered, e.g., for experimental, diagnostic, and/or therapeutic purposes. Subjects of interest herein include animals, particularly agriculturally significant animals or companion animals (e.g., ruminants, cows, steer, sheep, goats, horses, swine, dogs, cats, rabbits, birds, fish, etc.), zoo animals (giraffe, bears, zebra, lions, tigers, etc.), reptiles, laboratory animals (e.g., mice, rats), mammals, primates, or humans.

The term “stable”, as used herein, refers to compounds that are not substantially altered when subjected to conditions to allow for their production, detection, and, in certain embodiments, their recovery, purification, and use for one or more of the purposes disclosed herein.

As used herein, the term “treatment” refers to the provision of any type of medical or surgical management to a subject. Treating can include, but is not limited to, administering a device or composition delineated herein to a subject. Treating is typically undertaken in an effort to alter the course of a disease, disorder, or undesirable condition in a manner beneficial to the subject. The effect of treating can generally include reversing, alleviating, reducing, delaying the onset of, inhibiting the progression of, and/or reducing the likelihood of occurrence of the disease, disorder, or condition to which such term applies, or one or more symptoms or manifestations of such disease, disorder or condition. A composition of this invention can be administered to a subject who has developed an injury or is at increased risk of developing an injury relative to a member of the general population. A composition of this invention can be administered prophylactically, i.e., before development of any symptom or manifestation of a condition. Typically in this case the subject will be at risk of developing the condition. The composition can be administered prior to exposure of the subject to activity that places the subject or subject body part at risk of injury or damage.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt. Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

The term “pharmaceutically acceptable,” as used herein, refers to a component that is suitable for use in contact with the humans and other mammals without undue toxicity, irritation, allergic response and the like, and are commensurate with a reasonable benefit/risk ratio.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The invention also provides compositions comprising an effective amount of a active chemical compound, or a pharmaceutically acceptable salt, solvate, hydrate, polymorph or prodrug, if applicable, of said compound; and an acceptable carrier. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in such compositions.

Other embodiments of the present invention include the following:

A method of treating a subject suffering from damage to a tendon comprising administering to the subject's tendon area an effective amount of a device to enhance one or more of blood flow, vasodilation, warmth or the sensation of warmth at the area.

A method of improving flexibility, elasticity, strength, healing, mobility, or comfort of a tendon in a subject comprising administering to the subject's tendon area an effective amount of a device to enhance one or more of blood flow, vasodilation, warmth or the sensation of warmth at the area.

A method of reducing or preventing damage to a tendon in a subject comprising administering to the subject's tendon area an effective amount of a device to enhance one or more of blood flow, vasodilation, warmth or the sensation of warmth at the area.

A method of reducing or preventing the risk of damage to a tendon in a subject during athletic activity comprising administering to the subject's tendon area prior to participation in the athletic activity an effective amount of a device to enhance one or more of blood flow, vasodilation, warmth or the sensation of warmth at the area.

A method of improving flexibility, elasticity, strength, healing, mobility, or comfort of a tendon in a subject comprising administering to the subject's tendon area an effective amount of a device that provides heat or the sensation of heat to the area for at least one hour (e.g., any number between 1 and 24 hours, inclusive) continuously.

A method of reducing or preventing damage to a tendon in a subject comprising administering to the subject's tendon area an effective amount of a device that provides heat or the sensation of heat to the area for at least one hour (e.g., any number between 1 and 24 hours, inclusive) continuously.

A method of reducing or preventing the risk of damage to a tendon in a subject during athletic activity comprising administering to the subject's tendon area prior to participation in the athletic activity an effective amount of a device that provides heat or the sensation of heat to the area for at least one hour (e.g., any number between 1 and 24 hours, inclusive) continuously.

A method of reducing pain and discomfort caused by damage to a tendon in a subject during athletic activity comprising administering to the subject's tendon area prior to participation in the athletic activity an effective amount of a device that provides heat or the sensation of heat to the area for at least one hour (e.g., any number between 1 and 24 hours, inclusive) continuously.

In other embodiments, the methods include any delineated herein:

wherein the device is a self adhesive patch having a composition including menthol, glyceryltrinitrate, or an iron-based exothermal reactant as the active agent;

wherein the device is applied for between 1 and 24 hours inclusive (e.g., 24 hours, 12 hours, 8 hours, 4 hours, any number between 1 and 24, inclusive, etc.);

wherein the device is applied for consecutive periods of between 1 and 8 hours each period;

wherein the subject suffers from microtrauma at the tendon;

wherein the subject suffers from repetitive stress injury (RSI) at the tendon;

wherein the subject suffers from a traumatic, exercise-related or overuse injury to the tendon. wherein the subject suffers from tendonitis, tendinosis, tendinopathy, retrocalcaneal bursitis, tendon tears, tendon rupture, tenosynovitis, degenerative tendon change, scar tissue formation, post-operative tendon repair, epicondylitis or epicondyalgia;

wherein the tendon is one or more of: the tendon of quadriceps, gracilis tendon, sartorius tendon, semitendinosis tendon, popliteus tendon, or adductor magnus tendon;

where the tendon is associated with one or more of the following injuries: achilles, rotator cuff, bicep tendon, tennis/golfer's elbow, patella tendon, hamstring, groin, tricep, ITB, or conditions of the tendons in the hand/wrist like DeQuervain's Tenosynovitis;

wherein the tendon is the Achilles tendon;

wherein administration of the device reduces the occurrence of excessive shortening or tightening of the Achilles tendon in the subject;

wherein the device is applied to the heel area of the foot; or

wherein the device provides a heating sensation to the tendon area.

Other aspects include:

A method of treating a subject suffering from damage to a tendon comprising administering to the subject's tendon area an effective amount of a device that provides heat or the sensation of heat to the area for at least one hour (e.g., any number between 1 and 24 hours, inclusive) continuously.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for topical administration. In certain embodiments, the compositions herein are administered transdermally (e.g., using a transdermal patch techniques). Other formulations may conveniently be presented in unit dosage form, e.g., sustained release formulations, and in liposomes, and may be prepared by any methods well known in the art of pharmacy. See, for example, Remington's Pharmaceutical Sciences, Mack Publishing Company, Philadelphia, PA (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and ionophoretic administration are also included in this invention. One of ordinary skill in the art would readily know how to determine doses and delivery formulation based on both the height and or weight of the patient.

The devices herein (e.g., wraps, patches, bandages, gauzes, braces) have associated with them a formulation for producing heat or a sensation of heat at the body area where the device and/or formulation is contacted. The formulations can include one or more, either alone or in combination, of agents that provide the sensation of heat, or actually provide heat to the body part of the subject, including for example, an analgesic (e.g., menthol, salicylate), or an iron-based exothermal reactant. In one embodiment, the invention relates to a liner, layer, substrate, material, etc. associated with the formulation, that is suitable for application using a brace (e.g., a disposable liner for a brace).

According to another embodiment, the invention provides a method of treating or preventing damage to a subject suffering from or susceptible to a disorder or injury that is beneficially treated by the compositions (or devices thereof) herein comprising the step of administering to said subject an effective amount of a composition or device of this invention. The present invention thus provides methods for the treatment or prevention of tendon injury disorders, or symptoms thereof (including those delineated herein) comprising the step of administering to a subject in need thereof at least one composition or device in an amount effective therefore. Other therapeutic agents such as those described below may be employed in the compositions in the present methods. In the methods of the present invention, such other therapeutic agent(s) may be administered prior to, simultaneously with or following the administration of the compositions(s)/device(s) of the present invention. Other aspects of the methods include those further comprising wherein a subject physical characteristic, metric or sample is assessed; assessing that is performed prior to administration of the compositions(s)/device(s); assessing that is performed after administration of the compositions(s)/device(s); or assessing that is performed both prior to and after administration of the compositions(s)/device(s).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic assessment or measurement of any physical attribute or metric, or sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., objective, subjective, or measurable characteristic) or diagnostic measurement (e.g., physical assessment) in a subject suffering from or susceptible to a disorder or symptoms thereof delineated herein, in which the subject has been administered a therapeutic amount of a compound or device herein sufficient to treat the disorder or symptoms thereof. The level of Marker determined in the method can be compared to known levels of Marker in either healthy normal controls or in other afflicted patients to establish the subject's disorder or injury status. In preferred embodiments, a second level of Marker in the subject is determined at a time point later than the determination of the first level, and the two levels are compared to monitor the course of the disorder or injury or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

In yet another aspect, the invention provides the use of a composition or device delineated herein (e.g., self-adhesive patch) alone or together with one or more of the above-described second therapeutic agents in the manufacture of a medicament or device, either as a single composition or as separate dosage forms, for treatment or prevention in a subject of a disease, disorder or symptom set forth above. Another aspect of the invention is a composition or device herein for use in the treatment or prevention in a subject of a disease, disorder or symptom thereof delineated herein.

Recitation of a numerical range includes each individual number in that range as well as is inclusive of the delineated beginning and ending numbers of the range. For example, a recitation of “n is independently 0-4” means n can be 0, 1, 2, 3 or 4 at any designated instance.

### EXAMPLES

### Example 1

### Devices

A. In one example, the device is a self-adhesive patch with a composition having menthol (1.4% or 5%) as the active agent and includes camphor, carboxymethylcellulose sodium, glycerin, kaolin, methyl acrylate/2-ethylhexyl acrylate copolymer, methyl salicylate, polyacrylic acid, polysorbate 80, sodium polyacrylate, tartaric acid, titanium dioxide, and water as additional agents. In an alternate version of the device, the composition has menthol present at a level of 5% and includes carboxymethylcellulose sodium, glycerin, kaolin, methyl acrylate/2-ethylhexyl acrylate copolymer, polyacrylic acid, polysorbate 80, sodium polyacrylate, tartaric acid, titanium dioxide, and water as additional agents.

B. In another example, the device is a wrap (primarily polypropylene) having an adhesive layer at parts of one face and having a plurality of cells, each cell including activated carbon, iron powder, sodium chloride, water and sodium thiosulfate pentahydrate as active components (i.e., heat generation reagents) therein. When exposed to air, the active components react to generate heat.

### Example 2

### Method for Treatment of Tendon Injury

A 42-year old male Caucasian subject incurred tendinitis damage to his Achilles tendon while participating in athletic activity. The subject was subjected to standard treatment regimens for such condition, including electrostimulation, ultrasound, light stretching, heat treatments (ca. 20 minutes or less per treatment), and massage. In each instance, no significant change in range of motion of the subject was observed after treatment, and the subject reported a lack of mobility, a lack of range of motion, and experiencing pain and stiffnessdespite the aforementioned treatments. Furthermore, the subject demonstrated a lack of leg strength in that he could not perform any two-legged calf raises The subject applied the device of Example 1 {5% menthol}to the Achilles area for periods of ca. 8 hours twice daily. After 1 day of application, the subject reported increased range of motion, increased mobility, decreased stiffness, reduced pain, and subsequently demonstrated improved leg strength by performing ca. 30 two-legged calf raises.

### Example 3

### Method for prevention of Tendon Injury

The subject was a 42-year old male Caucasian subject who had recovered from tendonitis damage to his Achilles tendon while participating in athletic activity. Upon resuming athletic activity, the subject attempted to play in a complete game of soccer, but was unable to play the entire game due to pain and discomfort at the Achilles tendon and could not run. Prior to another later soccer game, the subject applied the device of Example 1 (5% menthol) immediately prior to participation in athletic activity to the Achilles tendon. With the device applied, the subject was able to play an entire game and reported increased comfort during play, improved mobility, and the ability to run comfortably for the entire gameThe subject also reported that after playing in a game where the device was applied he encountered less pain than previously experienced after playing in a game where he had not applied the device prior to commencing play. Moreover, the application of the device pre-game also resulted in less inconvenience after the game (relative to games without pre-application of the device) caused by prolonged care and administration associated with heat/cold and other treatment protocols, thus resulting in resumption of “normal” family activity more quickly.

The recitation of a listing of elements in any definition of a variable herein includes definitions of that variable as any single element or combination of listed elements. The recitation of an element, or an embodiment herein includes that element or embodiment as any single element or embodiment or in combination with any other element, embodiments or portions thereof.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

What is claimed is:

1. A method of treating a subject suffering from damage to a tendon comprising administering to the subject's tendon area an effective amount of a device to enhance one or more of blood flow, vasodilation, warmth or the sensation of warmth at the area.

2. A method of improving flexibility, elasticity, strength, healing, mobility, or comfort of a tendon in a subject comprising administering to the subject's tendon area an effective amount of a device to enhance one or more of blood flow, vasodilation, warmth or the sensation of warmth at the area.

3. (canceled)

4. (canceled)

5. The method of claim 1, wherein the device is a self adhesive patch having a composition including menthol, glyceryltrinitrate, or an iron-based exothermal reactant as the active agent.

6. The method of claim 1, wherein the device is applied for between 1 and 24 hours inclusive.

7. The method of claim 1, wherein the device is applied for consecutive periods of between 1 and 8 hours each period.

8. The method of claim 1, wherein the subject suffers from microtrauma at the tendon.

9. The method of claim 1, the subject suffers from repetitive stress injury (RSI) at the tendon.

10. The method of claim 1, wherein the subject suffers from a traumatic, exercise-related or overuse injury to the tendon.

11. The method of claim 1, wherein the subject suffers from tendonitis, tendinosis, tendinopathy, retrocalcaneal bursitis, tendon tears, tendon rupture, tenosynovitis, degenerative tendon change, scar tissue formation, post-operative tendon repair, epicondylitis or epicondyalgia.

12. The method of claim 1, wherein the tendon is one or more of: the tendon of quadriceps, gracilis tendon, sartorius tendon, semitendinosis tendon, popliteus tendon, or adductor magnus tendon.

13. The method of claim 1, wherein the tendon is the Achilles tendon.

14. The method of claim 1, wherein administration of the device reduces the occurrence of excessive shortening or tightening of the Achilles tendon in the subject.

15. The method of claim 1, wherein the device is applied to the heel area of the foot.

16. The method of claim 1 wherein the device provides a heating sensation to the tendon area.

17. A method of treating a subject suffering from damage to a tendon comprising administering to the subject's tendon area an effective amount of a device that provides heat or the sensation of heat to the area for at least one hour continuously.

18. (canceled)

19. (canceled)

20. (canceled)

21. A method of reducing pain and discomfort caused by damage to a tendon in a subject during athletic activity comprising administering to the subject's tendon area prior to participation in the athletic activity an effective amount of a device that provides heat or the sensation of heat to the area for at least one hour continuously.
